(19) United States
(12) Patent Application Publication
(10) Pub. No.: US 2019/0120860 A1
(43) Pub. Date: Apr. 25, 2019

(54) METHOD FOR DETECTING BRAIN RELATED MALADIES USING OCULAR FLUID

(71) Applicant: Ascendant Dx, Springdale, AR (US)

(72) Inventors: Lindsay Rutherford, Fayetteville, AR (US); Anna Daily, Fayetteville, AR (US); Omid Moghadam, Fayetteville, AR (US)

(73) Assignee: Ascendant Dx, Springdale, AR (US)

(21) Appl. No.: 16/166,257

(22) Filed: Oct. 22, 2018

Related U.S. Application Data
(60) Provisional application No. 62/575,579, filed on Oct. 23, 2017.

(51) Int. Cl. G01N 33/68

(52) U.S. Cl. CPC G01N 33/6896; G01N 2800/60; G01N 2800/304

(57) ABSTRACT

The present disclosure is directed toward methods for detecting brain related maladies in a subject by measuring levels of at least one of the identified biomarkers, as compared to a control. Brain related maladies include physical trauma to the brain, neurodegenerative disorders, and mental illness. The detection of the target biomarkers in ocular fluid provides a simpler and more effective sample matrix as compared to other biological matrices.

### CROSS REFERENCE TO RELATED APPLICATIONS

This application claims the benefit of U.S. Provisional Patent Application No. 62/575,579, filed on Oct. 23, 2017, which is hereby incorporated by reference in its entirety.

### BACKGROUND

The present application encompasses diagnosing and/or monitoring a plurality of brain related maladies, including physical injuries, neurodegenerative disease, and mental health disorders, using biomarkers found in ocular fluid.

The human brain is a complex organ which controls every aspect of daily life. Physical injuries, degenerative diseases, and psychological disorders to and arising from the brain can have adverse effects on an individual's life. These brain related maladies can be generally broken down into three categories: physical trauma, neurodegenerative disease, and mental illness. Maladies from any one of these categories have been proven difficult to diagnose and treat therefore a reliable and fast diagnostic tool is needed in the trauma, psychological, and neurodegenerative treatment fields.

### Physical Trauma:

Physical trauma to the brain can cause a myriad of injuries ranging from minor bruising, or concussion, to internal bleeding, such as a hematoma. As such, the potential outcomes for the patient vary widely and can be greatly influenced by the speed and accuracy of proper diagnosis and treatment. The diagnosis of physical trauma induced brain injuries utilizes a series of cognitive and physiological assessments of symptoms and often times requires radiological imaging, such as a Computed Tomography (CT) scan or Magnetic resonance imaging (MM), which is not always readily available to ensure proper diagnosis is received. The broad range of injures from physical trauma to the brain can complicate proper diagnosing of a patient which inhibits fast and accurate treatment of these injuries, potentially resulting in negative outcomes for the patient.

### Mental Illness:

Mental illnesses affect the thinking, mood and/or behavior of a subject. Diagnosing mental illness is challenging with many having similar symptomology. Currently mental health practitioners primarily rely on consultations with patients to gain insight including review of family history, differential diagnosis, and review of current symptoms to propose a “most likely” diagnosis from an ever increasing plethora of mental health disorders. Proper diagnosis is the first step to effective treatment of any mental disorder and thus is a crucial steep. Mental illness symptoms can range from mild, requiring minimal treatment, to severe, requiring unwilling clinical intervention and treatment. Given the similarities between many forms of mental illness, a diagnostic test capable of differentiating different forms of mental illness is an invaluable tool for medical professionals in both the clinical and private practice settings.

### Neurodegenerative Disease:

Neurodegenerative diseases are progressive and incapacitating disorders affecting the neurons in the human brain leading to debilitating neurological deficits of a subjects' behavioral, cognitive and motor skills. (William W. Seeley, 2009) While many, if not all, of these diseases and disorders are incurable, treatment options are increased if the disease is caught early in its progression. (Friedlander, 2003) Currently many of the neurodegenerative diseases are diagnosed by differential diagnosis and occur later in the progression of the disease. In most, if not all cases, a disease diagnosis is not confirmed until post mortem. A diagnostic assay capable of diagnosing these maladies prior to the patient's death will provide additional treatment options for medical practitioners, ultimately improving the quality of life for the patients and potentially one day provide for a cure.

Diagnosis of brain maladies remains complicated and a simpler solution is greatly needed. Herein we outline a methodology to diagnose, monitor, or evaluate the treatment of brain maladies using biomarkers in ocular fluid. Ocular fluid provides a snapshot of the subject's health without the complex environment and numerous potential interferences observed in other biological samples including blood based samples. A quantitative assay for the detection of ocular fluid based biomarkers to diagnose, monitor treatment of, and determine efficacy of treatment of brain maladies is described herein.

### SUMMARY

Methods of diagnosing brain related maladies of a subject are provided herein. Methods for the characterization and diagnosis of physical brain trauma, mental illness, and neurodegenerative diseases are provided using biomarkers found in ocular fluid obtained from a subject suspected of, knowing to have, or being monitored for a brain related malady. Methods include obtaining a sample of ocular fluid from a subject and performing steps of detecting the level of at least one of the markers selected from the in table 1. The sample is optimally an ocular fluid sample, such as an isolated tear sample or ocular wash, but can also be another bodily fluid.

Kits for performing methods described herein along with medical devices capable of performing the diagnostic testing outlined are also provided herein.

### BRIEF DESCRIPTION OF THE DRAWINGS

FIG. 1 depicts a flow diagram outlining the method of the current invention to be used when a patient has a suspected brain related malady using biomarkers found in Table 1.

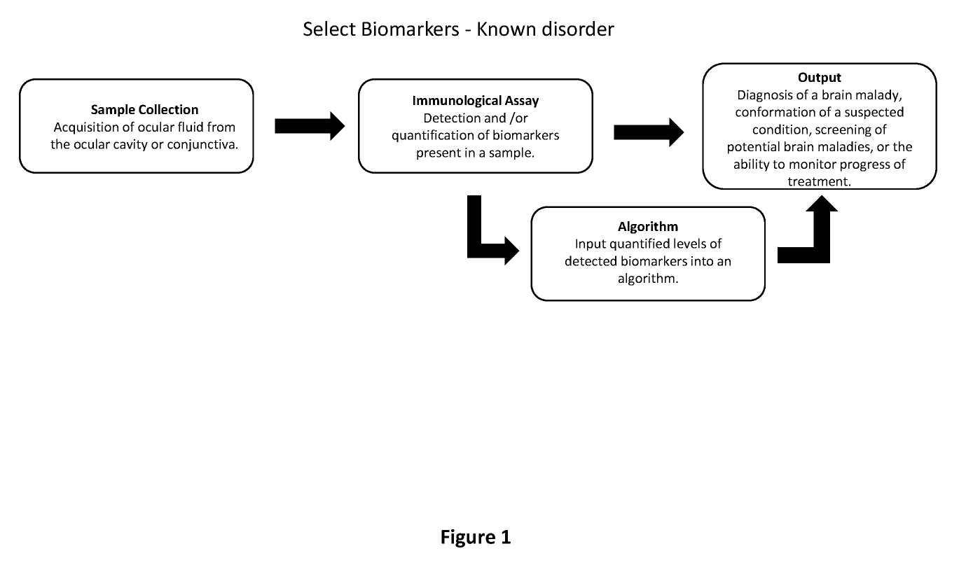

FIG. 2 depicts a flow diagram of the present invention in which the biomarkers found in Table 1 are used to screen a sample using a large number of said biomarkers at a given period.

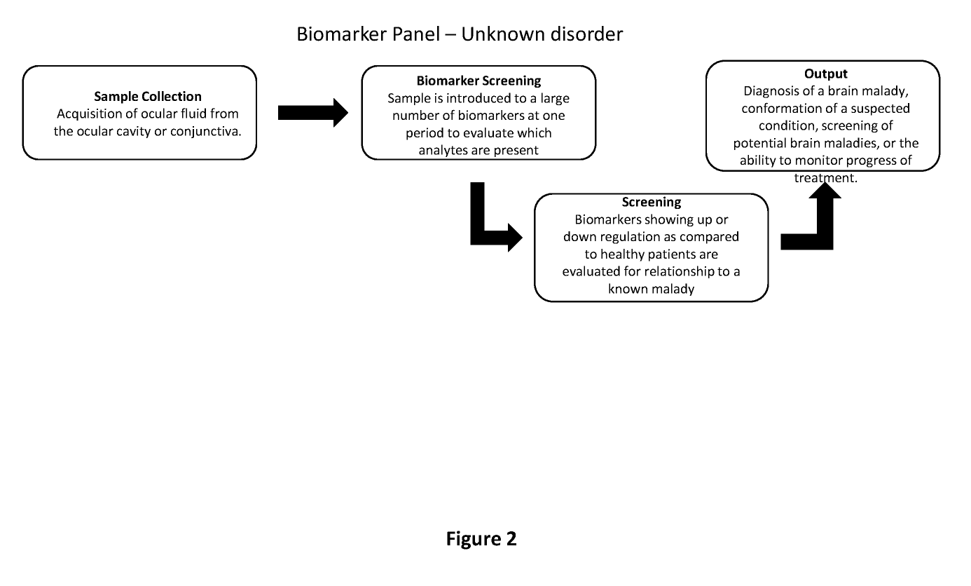

### DETAILED DESCRIPTION AND EMBODIMENTS

Provided herein is a method to diagnose brain related maladies using biomarkers found in ocular fluid. The biomarkers of interest for brain related maladies are selected from the following list and are also shown in Table 1: 14 kDa phosphohistidine phosphatase, 14-3-3 protein epsilon, 14-3-3 protein sigma, 14-3-3 protein theta, 26S protease regulatory subunit 6A, 2′-deoxynucleoside 5′-phosphate N-hydrolase 1, 40S ribosomal protein S28, 40S ribosomal protein S5, 40S ribosomal protein SA, 4-trimethylaminobutyraldehyde dehydrogenase, 60S acidic ribosomal protein P1, 6-phosphogluconate dehydrogenase, decarboxylating, 6-phosphogluconolactonase, 78 kDa glucose-regulated protein, Actin-related protein 2/3 complex subunit 1B, Actin-related protein 2/3 complex subunit 2, Actin-related protein 3, Acylamino-acid-releasing enzyme, Acyl-CoA-binding protein, Adenine phosphoribosyltransferase, Adenylate kinase isoenzyme 1, Adenylyl cyclase-associated protein 1, Adipogenesis regulatory factor, Adseverin, Afamin, Aflatoxin B1 aldehyde reductase member 2, Alcohol dehydrogenase [NADP(+)], Alcohol dehydrogenase 1C, Alcohol dehydrogenase class 4 mu/sigma chain, Aldehyde dehydrogenase family 1 member A3, Aldehyde dehydrogenase, dimeric NADP-preferring, Aldo-keto reductase family 1 member C1, Alpha_1_Antitrypsin, Alpha-1-acid glycoprotein 1, Alpha-1-antichymotrypsin, Alpha-1-antitrypsin, Alpha-1B-glycoprotein, Alpha2 Macroglobulin, Alpha-2-antiplasmin, Alpha-2-HS-glycoprotein, Alpha-2-macroglobulin, Alpha-actinin-4, Alpha-aminoadipic semialdehyde dehydrogenase, Alpha-amylase 1, Alpha-enolase, Aminopeptidase B, Angiotensinogen, Annexin A1, Annexin A11, Annexin A2, Annexin A3, Annexin A4, Annexin A5, Anterior gradient protein 2 homolog, Antileukoproteinase, Antithrombin-III, Apolipoprotein A-I, Apolipoprotein A-II, Apolipoprotein A-IV, apoliprotein A1, Argininosuccinate synthase, Aspartate aminotransferase, cytoplasmic, Astrocytic phosphoprotein PEA-15, Basement membrane-specific heparan sulfate proteoglycan core protein, BDNF, Beta-2-glycoprotein 1, Beta-2-microglobulin, Bifunctional purine biosynthesis protein PURH, Bone morphogenetic protein receptor type-2, Brain acid soluble protein 1, C4b-binding protein alpha chain, Calcyphosin, Calmodulin, Calmodulin-like protein 3, Calmodulin-like protein 5, Calpain small subunit 1, Calpain-1 catalytic subunit, Calpain-2 catalytic subunit, Calpastatin, Calreticulin, Calumenin, Carbonic anhydrase 13, Carbonyl reductase [NADPH] 1, Caspase-14, Catalase, Catechol O-methyltransferase, Cathepsin B, Cell division control protein 42 homolog, Ceruloplasmin, Charged multivesicular body protein 4b, Chitinase-3-like protein 2, Chloride intracellular channel protein 1, Chromosome 6 open reading frame 55, isoform CRA_b, Cluster of 14-3-3 protein zeta/delta, Cluster of Beta-actin-like protein 2, Cluster of CON_P08727, Cluster of Cystatin-S, Cluster of Extracellular glycoprotein lacritin, Cluster of Ezrin, Cluster of Fructose-bisphosphate aldolase A, Cluster of Haptoglobin, Cluster of Heat shock 70 kDa protein 1A/1B, Cluster of Heat shock protein beta-1, Cluster of Heat shock protein HSP 90-alpha, Cluster of Ig alpha-1 chain C region, Cluster of Ig alpha-1 chain V-III region HAH, Cluster of Ig gamma-1 chain C region, Cluster of Ig kappa chain V-I region EU, Cluster of Ig kappa chain V-III region HAH, Cluster of Ig lambda chain V-III region LOI, Cluster of Protein disulfide-isomerase A3, Cluster of Protein IGKV1-33, Cluster of Protein IGKV3-11, Cluster of Rab GDP dissociation inhibitor beta, Cluster of Serum albumin, Cluster of Tubulin beta-4B chain, Clusterin, Coagulation factor XII, Cofilin-1, Coiled-coil domain-containing protein 96, Complement C3, Complement C4-B, Complement component C7, Complement factor H, Complement factor I, Cornifin-B, Coronin-1A, Costars family protein ABRACL, Cullin-associated NEDD8-dissociated protein 1, Cystatin-B, Cystatin-C, Cystatin-D, Cystatin-SN, Cysteine-rich protein 1, Cytosol aminopeptidase, Cytosolic non-specific dipeptidase, D-3-phosphoglycerate dehydrogenase, D-dopachrome decarboxylase, Deleted in malignant brain tumors 1 protein, Dermcidin, Destrin, Dihydropteridine reductase, Dipeptidyl peptidase 3, DNA dC->dU-editing enzyme APOBEC-3A, Drebrin-like protein, Echinoderm microtubule-associated protein-like 2, EF-hand domain-containing protein D2, EGF, Elongation factor 1-alpha 1, Elongation factor 1-beta, Elongation factor 1-gamma, Elongation factor 2, Endoplasmic reticulum resident protein 29, Epidermal growth factor receptor kinase substrate 8-like protein 1, ERO1-like protein alpha, Ester hydrolase C11orf54, Eukaryotic initiation factor 4A-II, Eukaryotic translation initiation factor 4H, Eukaryotic translation initiation factor 6, F-actin-capping protein subunit alpha-1, F-actin-capping protein subunit alpha-2, Farnesyl diphosphate synthase, Fatty acid-binding protein, epidermal, F-box only protein 50, Fibrinogen alpha chain, Fibrinogen beta chain, Fibrinogen gamma chain, Filamin-B, Flavin reductase (NADPH), Fructose-1,6-bisphosphatase 1, defensin-1, Galectin-3, Galectin-3-binding protein, Gamma-glutamylcyclotransferase, GDP-L-fucose synthase, Gelsolin, Glucose-6-phosphate 1-dehydrogenase, Glucose-6-phosphate isomerase, Glutamine synthetase, Glutaredoxin-1, Glutathione reductase, mitochondrial, Glutathione S-transferase, Glutathione S-transferase P, Glutathione synthetase, Glyceraldehyde-3-phosphate dehydrogenase, Glycogen phosphorylase, liver form, Glyoxalase domain-containing protein 4, GMP reductase, Golgi membrane protein 1, GTP cyclohydrolase 1 feedback regulatory protein, GTP-binding nuclear protein Ran, Haptoglobulin, Heat shock 70 kDa protein 4, Heat shock cognate 71 kDa protein, Heme-binding protein 2, Hemoglobin subunit alpha, Hemoglobin subunit beta, Hemopexin, Heterogeneous nuclear ribonucleoprotein DO, Heterogeneous nuclear ribonucleoprotein K, Heterogeneous nuclear ribonucleoprotein M, Heterogeneous nuclear ribonucleoproteins A2/B1, High mobility group protein B1, Histidine triad nucleotide-binding protein 1, Histidine-rich glycoprotein, Histone H1.5, Histone H2A type 1-D, HLA class I histocompatibility antigen, B-54 alpha chain, Hsp90 co-chaperone Cdc37, Ig delta chain C region, Ig epsilon chain C region, Ig gamma-2 chain C region, Ig gamma-4 chain C region, Ig heavy chain V-III region TIL, Ig kappa chain V-I region BAN, Ig kappa chain V-IV region, Ig lambda chain V-I region HA, Ig lambda chain V-IV region Hil, Ig lambda-3 chain C regions, Ig mu chain C region, IL-6, Immunoglobulin J chain, Immunoglobulin lambda-like polypeptide 5, Importin subunit beta-1, Inorganic pyrophosphatase, Inositol polyphosphate 1-phosphatase, Insulin-like growth factor-binding protein complex acid labile subunit, Inter-alpha-trypsin inhibitor heavy chain H1, Inter-alpha-trypsin inhibitor heavy chain H2, Interleukin-1 receptor antagonist protein, Involucrin, Isocitrate dehydrogenase [NADP] cytoplasmic, ITIH4 protein, Keratin, Keratin type I cytoskeletal 9, Keratin type II cytoskeletal 1, Ketimine reductase mu-crystallin, Kininogen-1, Kynureninase, Lactoperoxidase, Lactotransferrin, Lactrin, Latexin, Leucine-rich alpha-2-glycoprotein, Leukocyte elastase inhibitor, Leukotriene A-4 hydrolase, LIM and SH3 domain protein 1, Lipocalin-1, Lipolysis-stimulated lipoprotein receptor, L-lactate dehydrogenase A chain, L-lactate dehydrogenase B chain, Low molecular weight phosphotyrosine protein phosphatase, Lymphocyte-specific protein 1, Lysine-tRNA ligase, Lysozyme C, Macrophage migration inhibitory factor, Macrophage-capping protein, Malate dehydrogenase, cytoplasmic, Malate dehydrogenase, mitochondrial, Mammaglobin-B, Matrix metalloproteinase-9, Mesothelin, Metalloproteinase inhibitor 1, MIP1beta, Mucin-SAC, Mucin-like protein 1, Myeloperoxidase, Myosin light polypeptide 6, Myosin regulatory light chain 12A, Myosin-14, Myosin-15, Myosin-9, Myotrophin, Na(+)/H(+) exchange regulatory cofactor NHE-RF1, N-acetylmuramoyl-L-alanine amidase, Nascent polypeptide-associated complex subunit alpha, muscle-specific form, Neutrophil defensin 1, Neutrophil gelatinase-associated lipocalin, Niban-like protein 1, Nicotinate phosphoribosyltransferase, Non-histone chromosomal protein HMG-14, Nucleobindin 2, isoform CRA_b, Nucleobindin-1, Nucleoside diphosphate kinase B, Omega-amidase NIT2, PAI-1, PDZ and LIM domain protein 1, PDZ and LIM domain protein 5, Peptidyl-prolyl cis-trans isomerase A, Peptidyl-prolyl cis-trans isomerase B, Peptidyl-prolyl cis-trans isomerase FKBP1A, Perilipin-3, Periplakin, Peroxiredoxin-1, Peroxiredoxin-2, Peroxiredoxin-5, mitochondrial, Peroxiredoxin-6, Phosphatidylethanolamine-binding protein 1, Phosphoglucomutase-1, Phosphoglycerate kinase 1, Phosphoglycerate mutase 1, Phospholipase A2, membrane associated, Phospholipid transfer protein, Plasma protease C1 inhibitor, Plasminogen, Plasminogen activator inhibitor 1 RNA-binding protein, Plastin-2, Plastin-3, Platelet-activating factor acetylhydrolase IB subunit beta, Plectin, Poly(rC)-binding protein 1, Polymeric immunoglobulin receptor, Profilin-1, Programmed cell death 6-interacting protein, Prolactin, Prolactin-inducible protein, Proline-rich protein 27, Proline-rich protein 4, Prolyl endopeptidase, Prosaposin, Prostasin, Proteasome activator complex subunit 1, Proteasome activator complex subunit 2, Proteasome subunit alpha type-4, Proteasome subunit alpha type-7, Proteasome subunit beta type-2, Protein AMBP, Protein deglycase DJ-1, Protein disulfide-isomerase, Protein IGHV3-49, Protein IGHV4-4, Protein IGKV2-28, Protein IGKV2-30, Protein IGKV2D-24, Protein IGLV3-19, Protein NDRG2, Protein S100-A11, Protein S100-A4, Protein S100-A6, Protein S100-A8, Protein S100-A9, Protein S100-P, Protein SETSIP, Protein-glutamine gamma-glutamyltransferase 2, Prothrombin, Prothymosin alpha, Puromycin-sensitive aminopeptidase, Putative uncharacterized protein FLJ37218, Pyruvate kinase PKM, RANTES, Ras GTPase-activating-like protein IQGAP1, Ras-related protein Rab-10, Ras-related protein Rab-1A, Ras-related protein Rab-7a, Renin, Reticulocalbin-1, Retinal dehydrogenase 1, Retinoic acid receptor responder protein 1, Retinol binding protein 4, plasma, isoform CRA_b, Rho GDP-dissociation inhibitor 1, Rho GDP-dissociation inhibitor 2, Ribonuclease 4, Ribonuclease inhibitor, Ribonuclease T2, Rootletin, S Protease regulatory subunit 6A, Secreted frizzled-related protein 1, Secretoglobin family 1D member 1, Selenium-binding protein 1, Serine hydroxymethyltransferase, cytosolic, Serine/threonine-protein phosphatase CPPED1, Serotransferrin, Serpin B5, SH3 domain-binding glutamic acid-rich-like protein, SH3 domain-binding glutamic acid-rich-like protein 3, Sialic acid synthase, Signal transducer and activator of transcription, Small proline-rich protein 2A, Small proline-rich protein 3, Small ubiquitin-related modifier 3, S-methyl-5′-thioadenosine phosphorylase, Sorbitol dehydrogenase, SPARC-like protein 1, Specifically androgen-regulated gene protein, Src substrate cortactin, Stem Cell Factor, Stress-induced-phosphoprotein 1, Submaxillary gland androgen-regulated protein 3B, Sulfhydryl oxidase 1, Sulfurtransferase, Superoxide dismutase [Cu—Zn], Superoxide dismutase [Mn], mitochondrial, Talin-1, Tax1-binding protein 3, T-complex protein 1 subunit beta, Thioredoxin, Thioredoxin domain-containing protein 17, Thioredoxin reductase 1, cytoplasmic, Thioredoxin-like protein 1 (Fragment), Thiosulfate sulfurtransferase/rhodanese-like domain-containing protein 1, Thymidine phosphorylase, Thymosin beta-10, Thymosin beta-4, TIMP1, TNF alpha receptor II, TNF-RII, Transaldolase, Transcobalamin-1, Transforming protein RhoA, Transgelin-2, Transitional endoplasmic reticulum ATPase, Transketolase, Transthyretin, Trefoil factor 3, Triosephosphate isomerase, Tropomyosin alpha-3 chain, Tropomyosin alpha-4 chain, Tryptophan-tRNA ligase, cytoplasmic, Tubulin-specific chaperone A, Ubiquitin-40S ribosomal protein S27a, Ubiquitin-like modifier-activating enzyme 1, UMP-CMP kinase, UTP-glucose-1-phosphate uridylyltransferase, UV excision repair protein RAD23 homolog B, VEGF, VEGF coregulated chemokine 1, Vimentin, Vitamin D-binding protein, Vitronectin, V-type proton ATPase subunit B, brain isoform, V-type proton ATPase subunit G 1, WD repeat-containing protein 1, Xaa-Pro aminopeptidase 1, Xaa-Pro dipeptidase, Zinc-alpha-2-glycoprotein, and Zymogen granule protein 16 homolog B.

The term “brain related malady”, as used throughout the present disclosure, is defined as any disorder of the brain caused by disease, injury, or natural aging which causes cognitive or physiological impairment of the subject. Brain related maladies can generally be classified into three categories each with differing causalities: Physical trauma, neurodegenerative disease, and mental health. Each category of brain disorder brings its own host of diagnostic issues making it difficult for physicians and medical professionals to diagnose. The present disclosure generally seeks to expedite diagnosis of the outlined brain related maladies through use of biomarkers found in ocular fluid obtained from a subject.

Research using biomarkers has grown significantly over recent years. Biomarkers have been shown to provide previously unthinkable insight into the overall health of a subject. As discussed by Daily et. al. biomarkers have been shown to diagnose various types of cancer including breast cancer. (U.S. patent application Ser. Nos. 14/879,982 and 14/707,089) Biomarkers are defined as a biological compound or molecule, including protein and protein fragments, found in a biological sample which is indicative of a normal or abnormal biological process. (NCI webpage). Biomarkers are found in most any biological medium including, blood, tissue, saliva, seamen, vaginal secretion, mucus, hair, spinal fluid, and plasma. The present invention utilizes ocular fluid as a diagnostic medium for biomarker quantification and detection. Ocular fluid, as referenced throughout this disclosure, is defined as any fluid or liquid obtained from any surface of the eye or ocular cavity containing components (protein, DNA, RNA, bacteria, viruses, etc.) which have arisen within the ocular cavity, including the lacrimal gland, meibomian gland, or other tissues that connect with the lymphatic system, or at another location within the body. The terms “ocular fluid”, “tear(s)”, “ocular secretion”, and “ocular wash” are used interchangeably herein. Examples of ocular fluid comprise lacrimal secretions, vitreous humor, aqueous humor, meibum, and tears.

TABLE 1

| # | Biomarkers |
|---|---|
| 1 | 14 kDa phosphohistidine phosphatase |
| 2 | 14-3-3 protein epsilon |
| 3 | 14-3-3 protein sigma |
| 4 | 14-3-3 protein theta |
| 5 | 26S protease regulatory subunit 6A |
| 6 | 2′-deoxynucleoside 5′-phosphate N- |
| | hydrolase 1 |
| 7 | 40S ribosomal protein S28 |
| 8 | 40S ribosomal protein S5 |
| 9 | 40S ribosomal protein SA |
| 10 | 4-trimethylaminobutyraldehyde |
| | dehydrogenase |
| 11 | 60S acidic ribosomal protein P1 |
| 12 | 6-phosphogluconate dehydrogenase, |
| | decarboxylating |
| 13 | 6-phosphogluconolactonase |
| 14 | 78 kDa glucose-regulated protein |
| 15 | Actin-related protein 2/3 complex subunit 1B |
| 16 | Actin-related protein 2/3 complex subunit 2 |
| 17 | Actin-related protein 3 |
| 18 | Acylamino-acid-releasing enzyme |
| 19 | Acyl-CoA-binding protein |
| 20 | Adenine phosphoribosyltransferase |
| 21 | Adenylate kinase isoenzyme 1 |
| 22 | Adenylyl cyclase-associated protein 1 |
| 23 | Adipogenesis regulatory factor |
| 24 | Adseverin |
| 25 | Afamin |
| 26 | Aflatoxin B1 aldehyde reductase member 2 |
| 27 | Alcohol dehydrogenase [NADP(+)] |
| 28 | Alcohol dehydrogenase 1C |
| 29 | Alcohol dehydrogenase class 4 mu/sigma |
| | chain |
| 30 | Aldehyde dehydrogenase family 1 member |
| | A3 |
| 31 | Aldehyde dehydrogenase, dimeric NADP- |
| | preferring |
| 32 | Aldo-keto reductase family 1 member C1 |
| 33 | Alpha_1_Antitrypsin |
| 34 | Alpha-1-acid glycoprotein 1 |
| 35 | Alpha-1-antichymotrypsin |
| 36 | Alpha-1-antitrypsin |
| 37 | Alpha-1B-glycoprotein |
| 38 | Alpha2 Macroglobulin |
| 39 | Alpha-2-antiplasmin |
| 40 | Alpha-2-HS-glycoprotein |
| 41 | Alpha-2-macroglobulin |
| 42 | Alpha-actinin-4 |
| 43 | Alpha-aminoadipic semialdehyde |
| | dehydrogenase |
| 44 | Alpha-amylase 1 |
| 45 | Alpha-enolase |
| 46 | Aminopeptidase B |
| 47 | Angiotensinogen |
| 48 | Annexin A1 |
| 49 | Annexin A11 |
| 50 | Annexin A2 |
| 51 | Annexin A3 |
| 52 | Annexin A4 |
| 53 | Annexin A5 |
| 54 | Anterior gradient protein 2 homolog |
| 55 | Antileukoproteinase |
| 56 | Antithrombin-III |
| 57 | Apolipoprotein A-I |
| 58 | Apolipoprotein A-II |
| 59 | Apolipoprotein A-IV |
| 60 | apoliprotein A1 |
| 61 | Argininosuccinate synthase |
| 62 | Aspartate aminotransferase, cytoplasmic |
| 63 | Astrocytic phosphoprotein PEA-15 |
| 64 | Basement membrane-specific heparan |
| | sulfate proteoglycan core protein |
| 65 | BDNF |
| 66 | Beta-2-glycoprotein 1 |
| 67 | Beta-2-microglobulin |
| 68 | Bifunctional purine biosynthesis protein |
| | PURH |
| 69 | Bone morphogenetic protein receptor type-2 |
| 70 | Brain acid soluble protein 1 |
| 71 | C4b-binding protein alpha chain |
| 72 | Calcyphosin |
| 73 | Calmodulin |
| 74 | Calmodulin-like protein 3 |
| 75 | Calmodulin-like protein 5 |
| 76 | Calpain small subunit 1 |
| 77 | Calpain-1 catalytic subunit |
| 78 | Calpain-2 catalytic subunit |
| 79 | Calpastatin |
| 80 | Calreticulin |
| 81 | Calumenin |
| 82 | Carbonic anhydrase 13 |
| 83 | Carbonyl reductase [NADPH] 1 |
| 84 | Caspase-14 |
| 85 | Catalase |
| 86 | Catechol O-methyltransferase |
| 87 | Cathepsin B |
| 88 | Cell division control protein 42 homolog |
| 89 | Ceruloplasmin |
| 90 | Charged multivesicular body protein 4b |
| 91 | Chitinase-3-like protein 2 |
| 92 | Chloride intracellular channel protein 1 |
| 93 | Chromosome 6 open reading frame 55, |
| | isoform CRA_b |
| 94 | Cluster of 14-3-3 protein zeta/delta |
| 95 | Cluster of Beta-actin-like protein 2 |
| 96 | Cluster of CON_{——}P08727 |
| 97 | Cluster of Cystatin-S |
| 98 | Cluster of Extracellular glycoprotein lacritin |
| 99 | Cluster of Ezrin |
| 100 | Cluster of Fructose-bisphosphate aldolase A |
| 101 | Cluster of Haptoglobin |
| 102 | Cluster of Heat shock 70 kDa protein 1A/1B |
| 103 | Cluster of Heat shock protein beta-1 |
| 104 | Cluster of Heat shock protein HSP 90-alpha |
| 105 | Cluster of Ig alpha-1 chain C region |
| 106 | Cluster of Ig alpha-1 chain V-III region HAH |
| 107 | Cluster of Ig gamma-1 chain C region |
| 108 | Cluster of Ig kappa chain V-I region EU |
| 109 | Cluster of Ig kappa chain V-III region HAH |
| 110 | Cluster of Ig lambda chain V-III region LOI |
| 111 | Cluster of Protein disulfide-isomerase A3 |
| 112 | Cluster of Protein IGKV1-33 |
| 113 | Cluster of Protein IGKV3-11 |
| 114 | Cluster of Rab GDP dissociation inhibitor beta |
| 115 | Cluster of Serum albumin |
| 116 | Cluster of Tubulin beta-4B chain |
| 117 | Clusterin |
| 118 | Coagulation factor XII |
| 119 | Cofilin-1 |
| 120 | Coiled-coil domain-containing protein 96 |
| 121 | Complement C3 |
| 122 | Complement C4-B |
| 123 | Complement component C7 |
| 124 | Complement factor H |
| 125 | Complement factor I |
| 126 | Cornifin-B |
| 127 | Coronin-1A |
| 128 | Costars family protein ABRACL |
| 129 | Cullin-associated NEDD8-dissociated protein 1 |
| 130 | Cystatin-B |
| 131 | Cystatin-C |
| 132 | Cystatin-D |
| 133 | Cystatin-SN |
| 134 | Cysteine-rich protein 1 |
| 135 | Cytosol aminopeptidase |
| 136 | Cytosolic non-specific dipeptidase |
| 137 | D-3-phosphoglycerate dehydrogenase |
| 138 | D-dopachrome decarboxylase |
| 139 | Defensin-1 |
| 140 | Deleted in malignant brain tumors 1 protein |
| 141 | Dermcidin |
| 142 | Destrin |
| 143 | Dihydropteridine reductase |
| 144 | Dipeptidyl peptidase 3 |
| 145 | DNA dC−>dU-editing enzyme APOBEC-3A |
| 146 | Drebrin-like protein |
| 147 | Echinoderm microtubule-associated protein-like 2 |
| 148 | EF-hand domain-containing protein D2 |
| 149 | EGF |
| 150 | Elongation factor 1-alpha 1 |
| 151 | Elongation factor 1-beta |
| 152 | Elongation factor 1-gamma |
| 153 | Elongation factor 2 |
| 154 | Endoplasmic reticulum resident protein 29 |
| 155 | Epidermal growth factor receptor kinase |
| | substrate 8-like protein 1 |
| 156 | ERO1-like protein alpha |
| 157 | Ester hydrolase C11orf54 |
| 158 | Eukaryotic initiation factor 4A-II |
| 159 | Eukaryotic translation initiation factor 4H |
| 160 | Eukaryotic translation initiation factor 6 |
| 161 | F-actin-capping protein subunit alpha-1 |
| 162 | F-actin-capping protein subunit alpha-2 |
| 163 | Farnesyl diphosphate synthase |
| 164 | Fatty acid-binding protein, epidermal |
| 165 | F-box only protein 50 |
| 166 | Fibrinogen alpha chain |
| 167 | Fibrinogen beta chain |
| 168 | Fibrinogen gamma chain |
| 169 | Filamin-B |
| 170 | Flavin reductase (NADPH) |
| 171 | Fructose-1,6-bisphosphatase 1 |
| 172 | Galectin-3 |
| 173 | Galectin-3-binding protein |
| 174 | Gamma-glutamylcyclotransferase |
| 175 | GDP-L-fucose synthase |
| 176 | Gelsolin |
| 177 | Glucose-6-phosphate 1-dehydrogenase |
| 178 | Glucose-6-phosphate isomerase |
| 179 | Glutamine synthetase |
| 180 | Glutaredoxin-1 |
| 181 | Glutathione reductase, mitochondrial |
| 182 | Glutathione S-transferase |
| 183 | Glutathione S-transferase P |
| 184 | Glutathione synthetase |
| 185 | Glyceraldehyde-3-phosphate dehydrogenase |
| 186 | Glycogen phosphorylase, liver form |
| 187 | Glyoxalase domain-containing protein 4 |
| 188 | GMP reductase |
| 189 | Golgi membrane protein 1 |
| 190 | GTP cyclohydrolase 1 feedback regulatory |
| | protein |
| 191 | GTP-binding nuclear protein Ran |
| 192 | Haptoglobulin |
| 193 | Heat shock 70 kDa protein 4 |
| 194 | Heat shock cognate 71 kDa protein |
| 195 | Heme-binding protein 2 |
| 196 | Hemoglobin subunit alpha |
| 197 | Hemoglobin subunit beta |
| 198 | Hemopexin |
| 199 | Heterogeneous nuclear ribonucleoprotein D0 |
| 200 | Heterogeneous nuclear ribonucleoprotein K |
| 201 | Heterogeneous nuclear ribonucleoprotein M |
| 202 | Heterogeneous nuclear ribonucleoproteins |
| | A2/B1 |
| 203 | High mobility group protein B1 |
| 204 | Histidine triad nucleotide-binding protein 1 |
| 205 | Histidine-rich glycoprotein |
| 206 | Histone H1.5 |
| 207 | Histone H2A type 1-D |
| 208 | HLA class I histocompatibility antigen, B-54 |
| | alpha chain |
| 209 | Hsp90 co-chaperone Cdc37 |
| 210 | Ig delta chain C region |
| 211 | Ig epsilon chain C region |
| 212 | Ig gamma-2 chain C region |
| 213 | Ig gamma-4 chain C region |
| 214 | Ig heavy chain V-III region TIL |
| 215 | Ig kappa chain V-I region BAN |
| 216 | Ig kappa chain V-IV region |
| 217 | Ig lambda chain V-I region HA |
| 218 | Ig lambda chain V-IV region Hil |
| 219 | Ig lambda-3 chain C regions |
| 220 | Ig mu chain C region |
| 221 | IL-6 |
| 222 | Immunoglobulin J chain |
| 223 | Immunoglobulin lambda-like polypeptide 5 |
| 224 | Importin subunit beta-1 |
| 225 | Inorganic pyrophosphatase |
| 226 | Inositol polyphosphate 1-phosphatase |
| 227 | Insulin-like growth factor-binding protein |
| | complex acid labile subunit |
| 228 | Inter-alpha-trypsin inhibitor heavy chain H1 |
| 229 | Inter-alpha-trypsin inhibitor heavy chain H2 |
| 230 | lnterleukin-1 receptor antagonist protein |
| 231 | Involucrin |
| 232 | Isocitrate dehydrogenase [NADP] |
| | cytoplasmic |
| 233 | ITIH4 protein |
| 234 | Keratin |
| 235 | Keratin type I cytoskeletal 9 |
| 236 | Keratin type II cytoskeletal 1 |
| 237 | Ketimine reductase mu-crystallin |
| 238 | Kininogen-1 |
| 239 | Kynureninase |
| 240 | Lactoperoxidase |
| 241 | Lactotransferrin |
| 242 | Lactrin |
| 243 | Latexin |
| 244 | Leucine-rich alpha-2-glycoprotein |
| 245 | Leukocyte elastase inhibitor |
| 246 | Leukotriene A-4 hydrolase |
| 247 | LIM and SH3 domain protein 1 |
| 248 | Lipocalin-1 |
| 249 | Lipolysis-stimulated lipoprotein receptor |
| 250 | L-lactate dehydrogenase A chain |
| 251 | L-lactate dehydrogenase B chain |
| 252 | Low molecular weight phosphotyrosine |
| | protein phosphatase |
| 253 | Lymphocyte-specific protein 1 |
| 254 | Lysine--tRNA ligase |
| 255 | Lysozyme C |
| 256 | Macrophage migration inhibitory factor |
| 257 | Macrophage-capping protein |
| 258 | Malate dehydrogenase, cytoplasmic |
| 259 | Malate dehydrogenase, mitochondrial |
| 260 | Mammaglobin-B |
| 261 | Matrix metalloproteinase-9 |
| 262 | Mesothelin |
| 263 | Metalloproteinase inhibitor 1 |
| 264 | MIP1beta |
| 265 | Mucin-5AC |
| 266 | Mucin-like protein 1 |
| 267 | Myeloperoxidase |
| 268 | Myosin light polypeptide 6 |
| 269 | Myosin regulatory light chain 12A |
| 270 | Myosin-14 |
| 271 | Myosin-15 |
| 272 | Myosin-9 |
| 273 | Myotrophin |
| 274 | Na(+)/H(+) exchange regulatory cofactor NHE- |
| | RF1 |
| 275 | N-acetylmuramoyl-L-alanine amidase |
| 276 | Nascent polypeptide-associated complex |
| | subunit alpha, muscle-specific form |
| 277 | Neutrophil defensin 1 |
| 278 | Neutrophil gelatinase-associated lipocalin |
| 279 | Niban-like protein 1 |
| 280 | Nicotinate phosphoribosyltransferase |
| 281 | Non-histone chromosomal protein HMG-14 |
| 282 | Nucleobindin 2, isoform CRA_b |
| 283 | Nucleobindin-1 |
| 284 | Nucleoside diphosphate kinase B |
| 285 | Omega-amidase NIT2 |
| 286 | PAI-1 |
| 287 | PDZ and LIM domain protein 1 |
| 288 | PDZ and LIM domain protein 5 |
| 289 | Peptidyl-prolyl cis-trans isomerase A |
| 290 | Peptidyl-prolyl cis-trans isomerase B |
| 291 | Peptidyl-prolyl cis-trans isomerase FKBP1A |
| 292 | Perilipin-3 |
| 293 | Periplakin |
| 294 | Peroxiredoxin-1 |
| 295 | Peroxiredoxin-2 |
| 296 | Peroxiredoxin-5, mitochondrial |
| 297 | Peroxiredoxin-6 |
| 298 | Phosphatidylethanolamine-binding protein 1 |
| 299 | Phosphoglucomutase-1 |
| 300 | Phosphoglycerate kinase 1 |
| 301 | Phosphoglycerate mutase 1 |
| 302 | Phospholipase A2, membrane associated |
| 303 | Phospholipid transfer protein |
| 304 | Plasma protease C1 inhibitor |
| 305 | Plasminogen |
| 307 | Plasminogen activator inhibitor 1 RNA- |
| | binding protein |
| 308 | Plastin-2 |
| 309 | Plastin-3 |
| 310 | Platelet-activating factor acetylhydrolase IB |
| | subunit beta |
| 311 | Plectin |
| 312 | Poly(rC)-binding protein 1 |
| 313 | Polymeric immunoglobulin receptor |
| 314 | Profilin-1 |
| 315 | Programmed cell death 6-interacting protein |
| 316 | Prolactin |
| 317 | Prolactin-inducible protein |
| 318 | Proline-rich protein 27 |
| 319 | Proline-rich protein 4 |
| 320 | Prolyl endopeptidase |
| 321 | Prosaposin |
| 322 | Prostasin |
| 323 | Proteasome activator complex subunit 1 |
| 324 | Proteasome activator complex subunit 2 |
| 325 | Proteasome subunit alpha type -4 |
| 326 | Proteasome subunit alpha type-7 |
| 327 | Proteasome subunit beta type-2 |
| 328 | Protein AMBP |
| 329 | Protein deglycase DJ-1 |
| 330 | Protein disulfide-isomerase |
| 331 | Protein IGHV3-49 |
| 332 | Protein IGHV4-4 |
| 333 | Protein IGKV2-28 |
| 334 | Protein IGKV2-30 |
| 335 | Protein IGKV2D-24 |
| 336 | Protein IGLV3-19 |
| 337 | Protein NDRG2 |
| 338 | Protein S100-A11 |
| 339 | Protein S100-A4 |
| 340 | Protein S100-A6 |
| 341 | Protein S100-A8 |
| 342 | Protein S100-A9 |
| 343 | Protein S100-P |
| 344 | Protein SETSIP |
| 345 | Protein-glutamine gamma- |
| | glutamyltransferase 2 |
| 346 | Prothrombin |
| 347 | Prothymosin alpha |
| 348 | Puromycin-sensitive aminopeptidase |
| 349 | Putative uncharacterized protein FLJ37218 |
| 350 | Pyruvate kinase PKM |
| 351 | RANTES |
| 352 | Ras GTPase-activating-like protein IQGAP1 |
| 353 | Ras-related protein Rab-10 |
| 354 | Ras-related protein Rab-1A |
| 355 | Ras-related protein Rab-7a |
| 356 | Renin |
| 357 | Reticulocalbin-1 |
| 358 | Retinal dehydrogenase 1 |
| 359 | Retinoic acid receptor responder protein 1 |
| 360 | Retinol binding protein 4, plasma, isoform |
| | CRA_b |
| 361 | Rho GDP-dissociation inhibitor 1 |
| 362 | Rho GDP-dissociation inhibitor 2 |
| 363 | Ribonuclease 4 |
| 364 | Ribonuclease inhibitor |
| 365 | Ribonuclease T2 |
| 366 | Rootletin |
| 367 | S Protease regulatory subunit 6A |
| 368 | Secreted frizzled-related protein 1 |
| 369 | Secretoglobin family 1D member 1 |
| 370 | Selenium-binding protein 1 |
| 371 | Serine hydroxymethyltransferase, cytosolic |
| 372 | Serine/threonine-protein phosphatase |
| | CPPED1 |
| 373 | Serotransferrin |
| 374 | Serpin B5 |
| 375 | SH3 domain-binding glutamic acid-rich-like |
| | protein |
| 376 | SH3 domain-binding glutamic acid-rich-like |
| | protein 3 |
| 377 | Sialic acid synthase |
| 378 | Signal transducer and activator of |
| | transcription |
| 379 | Small proline-rich protein 2A |
| 380 | Small proline-rich protein 3 |
| 381 | Small ubiquitin-related modifier 3 |
| 382 | S-methyl-5′-thioadenosine phosphorylase |
| 383 | Sorbitol dehydrogenase |
| 384 | SPARC-like protein 1 |
| 385 | Specifically androgen-regulated gene protein |
| 386 | Src substrate cortactin |
| 387 | Stem Cell Factor |
| 388 | Stress-induced-phosphoprotein 1 |
| 389 | Submaxillary gland androgen-regulated |
| | protein 3B |
| 390 | Sulfhydryl oxidase 1 |
| 391 | Sulfurtransferase |
| 392 | Superoxide dismutase [Cu—Zn] |
| 393 | Superoxide dismutase [Mn], mitochondrial |
| 394 | Talin-1 |
| 395 | Tax1-binding protein 3 |
| 396 | T-complex protein 1 subunit beta |
| 397 | Thioredoxin |
| 398 | Thioredoxin domain-containing protein 17 |
| 399 | Thioredoxin reductase 1, cytoplasmic |
| 400 | Thioredoxin-like protein 1 (Fragment) |
| 401 | Thiosulfate sulfurtransferase/rhodanese-like |
| | domain-containing protein 1 |
| 402 | Thymidine phosphorylase |
| 403 | Thymosin beta-10 |
| 404 | Thymosin beta-4 |
| 405 | TIMP1 |
| 406 | TNF alpha receptor II |
| 407 | TNF-RII |
| 408 | Transaldolase |
| 409 | Transcobalamin-1 |
| 410 | Transforming protein RhoA |
| 411 | Transgelin-2 |
| 412 | Transitional endoplasmic reticulum ATPase |
| 413 | Transketolase |
| 414 | Transthyretin |
| 415 | Trefoil factor 3 |
| 416 | Triosephosphate isomerase |
| 417 | Tropomyosin alpha-3 chain |
| 418 | Tropomyosin alpha-4 chain |
| 419 | Tryptophan--tRNA ligase, cytoplasmic |
| 420 | Tubulin-specific chaperone A |
| 421 | Ubiquitin-40S ribosomal protein S27a |
| 422 | Ubiquitin-like modifier-activating enzyme 1 |
| 423 | UMP-CMP kinase |
| 424 | UTP--glucose-1-phosphate |
| | uridylyltransferase |
| 425 | UV excision repair protein RAD23 homolog B |
| 426 | VEGF |
| 427 | VEGF coregulated chemokine 1 |
| 428 | Vimentin |
| 429 | Vitamin D-binding protein |
| 430 | Vitronectin |
| 431 | V-type proton ATPase subunit B, brain |
| | isoform |
| 432 | V-type proton ATPase subunit G 1 |
| 433 | WD repeat-containing protein 1 |
| 434 | Xaa-Pro aminopeptidase 1 |
| 435 | Xaa-Pro dipeptidase |
| 436 | Zinc-alpha-2-glycoprotein |
| 437 | Zymogen granule protein 16 homolog B |

Subjects include humans, livestock, domesticated animals such as cats, dogs, cows, pigs, or other animals susceptible to brain trauma, mental illness, or neurodegenerative disease or is being tested for having or is suspected to have suffered a physical brain injury, brain trauma, mental illness, or neurodegenerative disease or other communicable, mental, or transmittable disease. Thus the terms “subject” and “patient” is used interchangeably herein. The subjects can be suspected of having a medical condition, being treated for a medical condition, or being monitored post treatment for a medical condition including brain related maladies. The methods and kits described herein can be used to diagnose, monitor, and prevent the spread of disease and other medical conditions. Medical condition as used throughout is defined as any physical or mental condition requiring diagnosis or treatment by a medical professional.

Biomarkers are found in multiple sources throughout the living organism. Often researchers and medical professionals rely on biomarkers found in blood samples as there is a comfort with the blood sample matrix, as it has been used extensively for many years. However the use of blood, including plasma, platelets, and serum as a sample medium for biomarker diagnostics poses significant issues when looking to quantify or detect certain biomarkers. Small molecular weight biomarkers tend to be obscured by other constituents in the blood sample matrix masking their presence, thus rendering them useless for diagnostic applications. As shown in Table 2 below, biomarkers can be identified in both blood and ocular fluid mediums. Ocular fluid often provides a more suitable medium for biomarker analysis as there are fewer interferences and processing steps involved. Also, the risk to technicians handling the samples are minimized with ocular fluid based samples as ocular fluid is void of many pathogens found in blood samples.

Physical trauma to the brain can cause a wide range of symptoms including impaired vision, headache, nausea, and dizziness/vertigo. The results of physical trauma can range from mild, such as a concussion, to severe, as in Traumatic Brain Injury (TBI). In many cases the signs and symptoms of physical brain trauma do not manifest until sometime after the initial injury leading to increased risk of secondary injuries (ex, athlete returning to play and sustaining another injury to the brain). The proper diagnosis is critical for the patient to avoid long term negative outcomes. Traditionally medical professionals rely on imaging to aid in the diagnosis of suspected brain trauma along with neurological and cognitive testing. Current imaging methods include CT scans and MM imaging. However these imaging techniques are not without issue. Radiographic imaging methods require specialized facilities and equipment. These facilities also require specialized staff to operate said equipment. Unfortunately, these facilities are not always readily available and thus MRI and CT techniques are not always readily available to the medical practitioner. Brain trauma often occurs far away from medical facilities such as in war zones and in underdeveloped regions throughout the world. Neurological evaluations of a subject, including assessments of vision, hearing, balance, coordination, and reflexes often times don't signal significant trauma has occurred. In the case of Cogitative testing, the assessment is based on subject answers and can be manipulated by the subject to avoid a negative outcome to the assessment. This is especially prevalent among military and athletes who see suspected brain trauma as a negative hindrance, and even nuisance, to performing their job or sport. The present invention seeks to improve diagnostic accuracy, speed of diagnosis, and lessen the financial burned on medical systems and the patient through the use of a simple diagnostic test to diagnose, categorize, and/or monitor brain trauma using biomarkers found in ocular secretions of a subject known to suspected to have suffered brain trauma. Brain trauma includes, but is not limited to, Concussion, Traumatic Brain Injury, hemorrhage, Hematoma, Skull Fracture, Diffuse Axonal Injury, stroke, and Edema.

Neurodegenerative diseases are conditions which affect the neurons in the human brain causing neurological deficits. (Prusiner, 2001) While many, if not all of these diseases, are presently incurable, treatment options are increased if the disease is caught early in its progression. Some of these disorders often target the geriatric population but can manifest in all age groups and demographics. Unfortunately, diagnosis of neurodegenerative disorders often times is not confirmed until after the subject's death. Examples of neurodegenerative diseases include, but are not limited to, Alzheimer's disease, Amyotrophic lateral sclerosis (ALS), Dementia, Friedreich's ataxia, Huntington's disease, Lewy Body Disease, Motor neuron disease, Parkinson's disease, Parkinson's disease related disorders, Prion disease, Spinocerebellar ataxia (SCA), and Spinal muscular atrophy.

Diagnosing and treating mental health disorders is a complex process which often times takes a significant amount of time to provide and accurate diagnosis. Similarities in symptoms between multiple disorders can obscure the underlying malady delaying treatment or indicating incorrect treatment techniques. Treatment options vary for each individual disorder therefore obtaining an accurate early diagnosis will allow the clinician to select the most effective treatment regime, rather than a trial and error process which often reduces patient compliance. Mental health disorders cover a wide range of potential maladies as outlined in the “Diagnostic and Statistical Manual of Mental Disorders Fifth Edition” (DSM-5) including: Amphetamine Dependence, Anorexia nervosa, Anxiety disorder, Asperger's Disorder, Attention-deficit hyperactivity disorder (ADHD), Autism spectrum disorder, Binge Eating Disorder, Bipolar Disorder, Borderline Personality disorder, Bulimia Nervosa, Cognitive disorders, Delirium, Dementia, Depression, Dissociative Amnesia, Dissociative Disorders Dissociative Identity Disorder, Dyspareunia, Dyssomnias, Erectile Disorder, Generalized Anxiety disorder, Impulse Control Disorder, Major Depressive Disorder (MDD), Obsessive-compulsive disorder (OCD), Panic Disorder, Parasomnias, Personality Disorders, Pervasive Developmental Disorder, Postpartum Depression, Posttraumatic Stress Disorder (PTSD), Psychosis, Schizophrenia, Seasonal Affective Disorder, Social Anxiety, Somatoform Disorders, substance abuse, and Tourette's disorder. Given the broad range of maladies which fall under the auspices of mental disorders, as outlined in the DSM 5, the term “mental disorder” is a general term covering a large number of maladies. The meaning of “mental disorder” as used throughout this disclosure is defined as “clinically significant behavior or psychological syndrome or pattern that occurs in an individual and that is associated with present distress (e.g., a painful symptom) or disability (i.e., impairment in one of more important areas of functioning) or with a significantly increased risk of suffering death, pain, disability, or an important loss of freedom” as outlined in the Diagnostic and Statistical Manual of Mental Disorders Fourth Edition (DSM-4).

Provided herein is a method to use biomarkers obtained from ocular secretions in diagnostic applications for brain related maladies. The biomarkers of interest are provided in Table 1.

The biomarkers, comprised of the proteins and protein fragments, shown in table 1 are shown in the subsequent embodiments to either increase or decrease in ocular fluid samples of a subject compared to a control group composed of subjects known to be absent of a given medical malady or disorder. These biomarkers will be used to determine the disease sate of a patient or other subject.

In one embodiment, the present invention utilizes collected ocular fluid, obtained through the use of Schirmer Tear Flow Test Strips or other absorbing material which is capable of absorbing the fluid present on the surface of the eye along with cellular material from the Conjunctiva, as the sample media. After collection, the sample is subjected to analysis for the presence and/or quantification of biomarkers present. The specific biomarkers of interest are shown in Table 1. In this embodiment, the detection and/or quantification of target biomarkers present in the sample is measured utilizing Enzyme-linked Immunosorbent Assay (ELISA). The resulting biomarker concentration is compared to a threshold value determined based on analysis of control samples obtained from healthy donors. For some biomarkers, an increase in value as compared to control samples, or upregulated, signal presence of an aforementioned disorder or injury. Likewise for some biomarkers a decrease in value as compared to control samples, or downregulated, signal presence of a target disorder. For a given malady, single or multiple biomarkers could be required to provide a suitable level of selectivity, specificity, and sensitivity. The biomarkers can be analyzed in parallel with other analytes or could be performed with single or multiple analytes of interest. The number of biomarkers, outlined in Table 1, required to screen, diagnose, monitor, or quantify a given malady can range from two to greater than 50. Most preferably the number of biomarkers required for a positive indication is less than five.

In yet another embodiment, the present invention is used in a medical clinic, emergency room, hospital, pediatric office, athletic training room, battlefield hospital or, military installation by a nurse, doctor, medic, athletic trainer, parent, police officer, fireman, or Emergency Medical Technician (EMT). This is ideally used when a patient presents with a suspected but unknown brain related malady. In this embodiment a single sample is analyzed for a larger number of biomarkers at once. This allows for a screening of a large number of potential disorders in a short period of time which will expedite the treatment for the patient. Sample collection consists of the procedure outlined above. The isolated sample is then subjected to an immunological assay. In this embodiment, the assay looks at a large number of biomarkers at a given time, as the biomarkers of interest are unknown since the brain malady is not known but the patient is suspected of having one. The immunological assay can be conducted using a number of immunological techniques known in the field including, but not limited to, ELISA, lateral flow immunoassay, or protein microarray. Each biomarker concentration is measured independently. The results are compared to control values of a given biomarker in a healthy individual. Analysis of each biomarker analyzed will provide information as to what, if any, brain related maladies a patient has. Once each biomarker has been quantified for a given sample, the values will be compared to biomarker levels in patients with known brain maladies. Biomarkers showing increased or decreased concentrations in the sample as compared to established levels in healthy populations, upregulated or downregulated respectively, will yield information into possible brain maladies. For a given brain related condition, such as depression, a single biomarker may be sufficient to provide adequate specificity and sensitivity for an accurate diagnosis, however multiple biomarkers each showing specific up or down regulated properties may be required.

In another embodiment the aforementioned analysis of ocular fluids for biomarkers found in Table 1 can be used to first detect suspected brain trauma, as outlined above, and then quantify the severity of said traumatic injury. This is especially helpful for potential TBI or Concussion related injuries as early diagnosis or detection is beneficial for treatment of said disorders. The physical proximity of the ocular cavity to the brain allows for fast detection of trauma related biomarkers in the event of an injury or suspected injury. Conformation of a previous diagnosis is also provided by this invention as is the ability to monitor the progression of a given disorder with time based on changes in the quantified biomarker concentrations found in the ocular samples. For maladies such as CTE, which currently can only be diagnosed postmortem, the ability to diagnose prior to death allows for potentially lifesaving treatments.

In another embodiment, the detection or quantification of disorder related biomarkers outlined in Table 1 is accomplished through the use of an immunological assay such as lateral flow or protein microarray. In this embodiment, selected biomarkers are detected through the use of biomarker, or analyte, specific antibodies which bind to the biomarker, or analyte, of interest. In the preferred embodiment, a sandwich assay is performed in which the analyte is captured between two analyte specific antibodies. For both lateral flow and protein microarray applications one antibody, termed capture antibody, is immobilized on a substrate. Upon introduction of the sample either by direct application to the substrate, as in protein microarray, or via lateral flow through a nitrocellulose membrane, as in lateral flow assays, the immobilized antibodies bind to the analyte of interest, in the present case biomarkers found in Table 1, if they are present in the sample. A second analyte specific antibody is conjugated to a visual indicator to allow for visual quantification or detection of said analyte. This antibody equipped with an indicator molecule is often termed “conjugate” for lateral flow applications. The visual indicator is generally colloidal gold or latex microspheres. Other indicators of interest are colloidal nanocrystals, magnetic nanoparticles, and organic dyes. The indicator preferably is colored or emits light in the visible, ultra-violet, or infra-red region of the electromagnetic spectrum. Fluorescent indicators are also if interest and can be utilized in this embodiment, especially for protein microarray. A lateral flow assay in which the biomarkers from Table 1 are detected or quantified has significant application in the emergency medical community as a fast and easy diagnostic tool.

In another embodiment, the biomarkers, taken singularly or in multiples, from those listed in table 1 are quantified in a given sample using a method previously described (generally an immunoassay). The results of said quantification is input into a mathematical algorithm. The output of said algorithm is a numerical likelihood that the subject has or does not have the brain related malady of interest. The aforementioned algorithm is developed by comparing the levels of each biomarker of interest to those found in samples taken from subjects known to be free of the brain related malady of interest. In some cases the levels of biomarkers detected will be upregulated, or increased, compared to control samples (samples taken from subjects known to be free of the target brain related malady or disorder). In other cases the levels of biomarkers of interest are down regulated, or lower than those of a control sample. In addition to quantified values of biomarker concentrations, other variables can be incorporated into said algorithm, such as age, family history, previous medical diagnosis, and other medical conditions. It would be obvious to one ordinary skilled in the art that this list is not all encompassing but rather representative examples of additional variable related to the subject which could be incorporated into said algorithm.

Treating brain related maladies includes, but is not limited to, reducing the number of symptoms, reducing the duration of symptoms, or reducing the need for further treatment in a subject. Treating a subject, as used herein, refers to any type of treatment that imparts a benefit to a subject afflicted with a disease or at risk of developing the disease, including improvement in the condition of the subject for example in one or more symptoms, delay in the progression of the disease, delay in the onset of symptoms, or delay in the progression of symptoms, etc.

The methods of the versions of this invention include detecting at least one biomarker. However, any number of biomarkers can be detected. It is preferred that at least two biomarkers are detected in the analysis. However, it is realized that three, four, or more, including all, of the biomarkers described herein can be utilized in the analysis. Thus, not only can one or more markers be detected, any number or combination of markers can be used in detection. In addition, other biomarkers not herein described can be combined with any of the presently disclosed biomarkers to aid in the diagnosis of a brain related malady. Moreover, any combination of the above biomarkers can be detected in accordance with versions of the present invention.

### Example 1

### Methods and Results for Label Free Quantitation by LC MS/MS

In solution trypsin digestion followed by LC MS/MS was carried out on 25 breast cancer samples, 25 benign samples, and 25 control samples by the Proteomic Core at the University of Arkansas for Medical Sciences (UAMS). Solution digests were carried out on all 75 samples in 100 mM ammonium bicarbonate (SigmaAldrich), following reduction in 10 mM Tris[2-carboxyethyl]phosphine (Pierce) and alkylation in 50 mM iodoacetamide (Sigma-Aldrich), by addition of 100 ng porcine trypsin (Promega) and incubation at 37° C. for 12-16 hours. Peptide products were then acidified in 0.1% formic acid (Fiuka). Tryptic peptides were separated by reverse phase Jupiter Proteo resin (Phenomenex) on a 100×0.075 mm column using a nanoAcquity UPLC system (Waters). Peptides were eluted using an 80 min gradient from 97:3 to 35:65 buffer A:B ratio. [Buffer A=0.1% formic acid, 0.05% acetonitrile; buffer B=0.1% formic acid, 75% acetonitrile.] Eluted peptides were ionized by electrospray (1.8 kV) followed by MS/MS analysis using collision-induced dissociation on an LTQ Orbitrap Velos mass spectrometer (Thermo). MS data were acquired using the FTMS analyzer in profile mode at a resolution of 60,000 over a range of 375 to 1500 m/z.

MS/MS data were acquired for the top 15 peaks from each MS scan using the ion trap analyzer in centroid mode and normal mass range with a normalized collision energy of 35.0. Proteins were identified from MS/MS spectra by database searching the Mascot search engine (Matrix Science) or MaxQuant quantitative proteomics software (Max Planck′ Institute). Mascot search results were compiled using Scaffold (Proteome Software). The following criteria were set to select a group of proteins that can be indicative of altered medical state of interest: 1) protein has a fold change of 1.5 or greater (in either positive or negative direction with respect to cancer). 2) fold change should be accompanied by p value of <0.05. 3) protein is present in 12 out of the 25 cancer samples. Using these criteria, the list of over 500 was reduced to the proteins found in Table 1.

### Example 2

Ocular fluid and blood samples were compared using exploratory microarray panels, a high-throughput ELISA based antibody array that gives qualitative/semi-quantitative protein expression profiling. This panel compared biomarker concentrations in ocular fluid with blood samples from the same subject to determine a fold change for over 500 proteins. Using traditional venipuncture procedures, 12 mls of blood was collected using an EDTA containing Vacutainer® (purple top tube). The sample was centrifuged for 15 minutes at 2500 RPM. The plasma was removed from the sample and placed into a sterile vial. Ocular fluid was collected using a Schirmer tear flow test strip (Ophthalmic diagnostic strip), the Schirmer strip was placed into a patient's lower eyelid for 5 minutes. The Schirmer was then removed and placed into a sterile vial containing buffer.

Two different types of arrays were used for this study, the first one detecting biomarkers associated with various cancer types, while the second detecting a wide range of biomarkers from multiple signaling pathways. Both of these arrays require 80-150 μg of protein, easily obtained from blood (plasma) or ocular fluid. Samples were placed onto microarray slides containing antibodies to proteins of interest, after incubation and washing the slides a fluorescent labeling dye was used as detection. The slides were scanned using a microarray scanner and analysis is achieved using image quantification software. Fold changes were determined by comparing each protein on the slides (ocular fluid array compared to plasma array). Results of selected biomarkers are shown in Table 2 demonstrating the increased fold change in ocular fluid over blood for a number of biomarkers of interest in brain related maladies.

TABLE 2
Biomarkers observed in Brain Maladies

| Biomarker | Tear:Blood¹ | Disease/Disorder |
|---|---|---|
| TIMP1 | 1.21 | Major depressive disorder² |
| IL-6 | 1.15 | Depression³ |
| Metalloproteinase -7 | 1.26 | Bipolar disorder⁴ |
| Insulin | 1.06 | Major depressive disorder² |
| Prolactin | 0.82 (receptor 9.19) | Major depressive disorder⁵ |
| TNF alpha receptor II | 1.09 | Major depressive disorder⁵ |

¹Data obtained by Inventors
²Dominca(2010)
³Jehn(2006)
⁴Fryer(2015
⁵Papakostas(2013)

It will be appreciated by those persons skilled in the art that variations and/or alterations could be made to the invention without varying from the scope or spirt of the present inventions described broadly. The presented embodiments are to be considered in all aspects as illustrative and not restrictive. All references cited herein are incorporated by reference to the maximum extent allowable by law.

### REFERENCES

American Psychiatric Association. (2013). Diagnostic and statistical manual of menial disorders (DSM-5®). American Psychiatric Pub.
American Psychiatric Association, 2000). Diagnostic and statistical manual of mental disorders (revised 4th ed.). American Psychiatric Pub
Daily, A., Rutherford, L. (2015). U.S. patent application Ser. No. 14/879,982.
Daily, A., Rutherford, L. (2015). U.S. patent application Ser. No. 14/707,089
Domenici, Enrico, Wille, D. R., Tozzi, F. Propokpenko, I., Miller, S., McKeown, A., Brittain, C., Rujescu, D., giegling, I., Turck, C., Holsboer, F., Bullmore, E., Middleton, L., Merlo-Pich, E., Alexander, R., Muglia, P., (2010) Plasma Protein Biomarkers for Depression and Schizophrenia by Multi Analyte Profiling of Case-Control Collections. PLOS ONE, 5(2), e9166.
Friedlander, R. M. (2003). Apoptosis and Caspases in Neurodegenerative Diseases. New England Journal of Medicine, 348(14), 1365-1375.
Frye, M. A., Nassan, M., Jenkins, G D., Kung., S., Veldic, M., Palmer, B A., Feeder, S E., Tye, S J., Choi, D S., Biernacka, J M., (2015). Feasibility of investigating differential proteomic expression in depression: implications for biomarker development in mood disorders., Transl Psychiatry. 5. 1-5.
Jehn, C F., Kuehnhardt, D., Bartholomae, A., Pfeiffer, S., Krebs, M., Regierer, A. C., Schmid, P., Possinger, K., Flath, B. C., (2006), Biomarkers of Depression in Cancer Patients., Cancer 107(11), 2723-2729.
Papakostas, G I., Shelton, R C., Kinrys, G., Henry, M E., Bakow, B R., Lipkin, S H., Pi, B., Thurmond, L., Bilello, J A. (2013), Assessment of a multi-assay, serum-based biological diagnostic test for major depressive disorder: a Pilot and Replication Study. Molecular Psychiatry, 18, 332-339.
Prusiner, S. B. (2001). Neurodegenerative Diseases and Prions. New England Journal of Medicine, 344(20), 1516-1526.
Seeley, W, Crawford, R. K., Zhou, J, Miller, B. L., Greicius, M. (2009). Neurodegenerative Diseases Target Large-Scale Human Brain Networks. Neuron, 62(1), 42-52.

What is claimed:

1. A method of determining if a subject has a brain related disorder comprising:
a. obtaining a sample of ocular fluid from a subject,
b. measuring levels in the sample of at least one of the biomarkers from the list provided in Table 1,
c. determining if the subject has, or is likely to have, a brain related disorder if the levels of the biomarker change as compared to levels in a control sample known to be absent of said brain related disorder.

2. The method of claim 1, wherein the brain related disorder is caused by a physical injury to the head or brain.

3. The method of claim 1, wherein the brain related disorder is a neurodegenerative disease.

4. The method of claim 1, wherein the brain related disorder is a mental illness.

5. The method of claim 1, wherein the subject is human.

6. The method of claim 1, wherein the subject is known to have a brain related disorder.

7. The method of claim 1 wherein said subject is being monitored for reoccurrence of said brain related disorder

8. The method of claim 1, wherein said subject is being evaluated for efficacy of a medical treatment for said brain related disorder.

9. The method of claim 1, wherein the biomarkers are decreased at least 1.5 fold, 2 fold, 4 fold, 8 fold or more relative to the level of said control sample.

10. The method of claim 1, wherein the biomarkers are increased at least 1.5 fold, 2 fold, 4 fold, 8 fold, or more relative to the level of said control sample.

11. The method of claim 1, wherein a portion of the biomarkers are increased at least 1.5 fold, 2 fold, 4 fold, 8 fold or more relative to the level of said control sample and a portion of the biomarkers are decreased at least 1.5 fold, 2 fold, 4 fold, 8 fold or more relative to the level of said control sample.

12. The method of claim 1 wherein a combination of biomarkers taken from Table 1 are used to determine the likelihood a subject has a brain related disorder.

13. The method of claim 1 wherein the level of the marker is detected by an antibody-based detection method or mass spectrometry.

14. The method of claim 1 wherein the level of biomarker is detected by multiplex protein detection method.

15. The method of claim 1, wherein the subject is at an increased risk of having or developing a brain related disorder.

16. The method of claim 1, wherein at least 3 biomarkers are used in combination

17. The method of claim 1 wherein at least 5 biomarkers are used in combination.

18. The method of claim 1, wherein at least 10 biomarkers are used in combination.

19. The method of claim 1 wherein determined levels of said biomarker or biomarkers are input into an algorithm.

20. The algorithm of claim 19 wherein the output from said algorithm is a probability a subject has the brain related disorder of interest.
